(19) United States
(12) Patent Application Publication
(10) Pub. No.: US 2026/0232725 A1
(43) Pub. Date: Aug. 13, 2026

(54) Method and Composition for the Inhalation of Hypochlorous Acid (HOCl) for Respiratory Treatment and Pathogen Elimination

(71) Applicant: Cyrus Darius Contractor, LENEXA, KS (US)

(72) Inventor: Cyrus Darius Contractor, LENEXA, KS (US)

(21) Appl. No.: 19/052,273

(22) Filed: Feb. 12, 2025

(51) Int. Cl. A61K 33/20; A61K 9/00

(52) U.S. Cl. CPC A61K 33/20; A61K 9/0078

(57) ABSTRACT

This invention provides a method for the therapeutic inhalation of hypochlorous acid (HOCl) to treat respiratory infections, reduce lung inflammation, and eliminate airborne pathogens in humans and animals. It describes controlled delivery of HOCl (10-50 ppm) via nebulizers, humidifiers, CPAP devices, ventilators, and direct tracheal instillation to improve oxygenation, break down mucus, and prevent secondary infections. The stabilized HOCl formulation ensures safety and efficacy, distinguishing it from surface disinfection applications. The method leverages HOCl's natural antimicrobial, anti-inflammatory, and mucolytic properties for direct respiratory treatment without toxicity or lung irritation. Applications include treating pneumonia, COVID-19, asthma, bronchitis, and ventilator-associated infections, as well as reducing airborne pathogen transmission in enclosed spaces. The invention presents a novel, non-toxic, and cost-effective approach to respiratory care, enhancing both individual treatment and public health disease mitigation.

### FIELD OF THE INVENTION

This invention relates to the therapeutic inhalation of hypochlorous acid (HOCl) for the treatment and prevention of respiratory infections, lung inflammation, and airborne pathogen mitigation. Specifically, it describes methods of delivering HOCl in controlled concentrations via inhalation to target respiratory conditions, airway inflammation, and infectious diseases, distinguishing it from surface disinfection applications.

### BACKGROUND OF THE INVENTION

Respiratory infections are a leading cause of morbidity and mortality worldwide. Existing treatments rely on antibiotics, antivirals, steroids, and supplemental oxygen, which may be ineffective in severe cases such as COVID-19, pneumonia, bronchitis, tuberculosis, and COPD exacerbations.

Hypochlorous acid (HOCl) is a naturally occurring antimicrobial agent produced by white blood cells to combat infections. It has well-documented bactericidal, virucidal, anti-inflammatory, and mucolytic properties. However, its direct application via inhalation for respiratory treatment remains underexplored in patent literature. This invention provides a safe, effective, and non-toxic method for the controlled inhalation of HOCl to treat respiratory illnesses, improve oxygenation, and reduce inflammation without causing lung irritation or toxicity.

### SUMMARY OF THE INVENTION

This invention provides a method for administering HOCl via inhalation to:
1. Reduce respiratory infections and airborne pathogen transmission.
2. Alleviate inflammation and improve oxygenation in the lungs.
3. Break down mucus and biofilms in the respiratory tract.
4. Deliver HOCl using controlled concentrations (10-50 ppm) through nebulizers, humidifiers, CPAP devices, ventilators, and other respiratory support systems.
5. Ensure formulation stability and delivery optimization for maximum therapeutic benefits.

### DETAILED DESCRIPTION OF THE INVENTION

### 1. HOCl Formulation for Inhalation

HOCl is electrolytically generated and stabilized within a pH range of 5.5 to 6.5, ensuring maximum antimicrobial potency while maintaining lung safety. pH may vary depending on each user.
The HOCl concentration must be precisely controlled within an effective inhalation range of 10-50 ppm to prevent airway irritation. This range will vary depending on user.

### 2. Methods of HOCl Inhalation Administration

### A. Nebulization

Method: A mesh or ultrasonic nebulizer disperses HOCl (10-50 ppm) into fine mist particles (<5 μm) for direct inhalation into the lungs.
Applications:
   Treatment for sinus infections, pneumonia, bronchitis, COVID-19, asthma, and COPD.
   Breakdown of mucus and biofilms to clear airways.

### B. HOCl Humidification (Continuous Low-Dose Inhalation)

Method: A cool-mist humidifier releases HOCl (10-20 ppm) into ambient air for passive inhalation.
Application:
   Airborne pathogen reduction in enclosed spaces (e.g., hospitals, airplanes, homes).
Preventative therapy for high-risk individuals (e.g., elderly, immunocompromised patients).

### C. CPAP & Oxygen Therapy Integration

Method: A humidifier chamber in CPAP or oxygen therapy devices is filled with HOCl (10-20 ppm) to sanitize airways during sleep.
Applications:
   Prevents bacterial colonization in CPAP users.
   Reduces lung inflammation in oxygen-dependent patients.

### D. Ventilator HOCl Administration for ICU Patients

Method: Sterile HOCl mist (10-20 ppm) is introduced via ventilator circuits for critically ill patients.
Applications:
   COVID-19 and pneumonia patients on ventilators.
   Reduction of secondary bacterial infections in ICU settings.

### E. Direct Tracheal Instillation (Emergency Use for Severe Cases)

Method: Sterile HOCl (10-20 ppm) is introduced directly into the trachea for patients with severe lung infections.
Applications:
   Emergency treatment of ventilator-associated pneumonia (VAP).
   Prevention of mucus buildup and airway obstruction.

1. A continuous positive airway pressure (CPAP) system configured to deliver hypochlorous acid (HOCl) to a human subject, comprising:
(a) an airflow generator configured to produce pressurized air;
(b) a conduit configured to deliver the pressurized air to a mask interface;
(c) an inline delivery module positioned within the conduit downstream of the airflow generator and upstream of the mask interface; and
(d) a dosing mechanism configured to introduce HOCl into the airflow within the inline delivery module at a controlled concentration for inhalation by the subject, wherein the HOCl is introduced into the airflow independently of a humidifier reservoir and is maintained within a predefined concentration range suitable for human inhalation.

2. The system of claim 1, wherein the dosing mechanism maintains the HOCl concentration within a range of about 10 to 50 parts per million in the airflow delivered to the subject.

3. The system of claim 1, wherein the inline delivery module comprises an aerosolization component configured to generate HOCl particles having a size of less than or equal to 5 microns.

4. The system of claim 1, wherein the dosing mechanism comprises a flow-controlled injection system configured to adjust HOCl delivery based on airflow rate within the conduit.

5. The system of claim 1, wherein the HOCl is stored in a sealed cartridge separate from the airflow conduit and introduced into the inline delivery module upon activation.

6. A method for delivering hypochlorous acid (HOCl) to a respiratory system of a human subject using a CPAP system, comprising: positioning an inline delivery module within an airflow conduit of the CPAP system downstream of an airflow generator and upstream of a mask interface; introducing HOCl from a sealed source into the inline delivery module independently of a humidifier reservoir; and controlling delivery of the HOCl into the airflow at a concentration of about 10 to 50 parts per million for inhalation by the subject.

7. A method for treating, preventing, or reducing vulvovaginal candidiasis, bacterial vaginosis, or associated vaginal malodor in a human subject, comprising topically applying to vaginal mucosal tissue a composition comprising stabilized hypochlorous acid (HOCl) at a concentration of about 10 to 200 parts per million and buffered to a pH of about 3.5 to 5.5, wherein the composition reduces one or more pathogenic microorganisms including Candida albicans within about two minutes of contact while maintaining viability of Lactobacillus species present in the vaginal microbiome.

8. The system of claim 1, wherein the inline delivery module further comprises a sensor configured to measure hypochlorous acid (HOCl) concentration in the airflow and provide feedback to the dosing mechanism to maintain the HOCl concentration within the predefined range.

9. A composition for vaginal application comprising stabilized hypochlorous acid (HOCl) in a pharmaceutically acceptable carrier, wherein the HOCl is present at a concentration of about 10 to 200 parts per million and buffered to a pH of about 3.5 to 5.5, and wherein the composition is formulated as a spray, gel, rinse, suppository, or wipe for topical application to vaginal mucosal tissue.

10. The system of claim 2, wherein the HOCl concentration is maintained within a range of about 10 to 30 parts per million in the airflow delivered to the subject.
